(19) United States
(12) Patent Application Publication
(10) Pub. No.: US 2005/0119693 A1
(43) Pub. Date: Jun. 2, 2005

(54) Medical forceps

(75) Inventor: Stephan Prestel, Rheinstetten-Morsch (DE)

Correspondence Address: COHEN, PONTANI, LIEBERMAN & PAVANE, 551 FIFTH AVENUE, SUITE 1210, NEW YORK, NY, 10176, US

(73) Assignee: Richard Wolf GmbH

(21) Appl. No.: 10/995,723

(22) Filed: Nov. 22, 2004

(30) Foreign Application Priority Data
Dec. 2, 2003 (DE) 203 18 591.9

(51) Int. Cl. A61B 17/28

(52) U.S. Cl. 606/207; 606/205; 606/208

(57) ABSTRACT

A medical forceps includes a shank having a distal end with a fixed jaw part, a movable jaw part which can pivot with respect to the fixed jaw part, and an actuation rod having a distal end which is axially movable with respect to the shank in order to pivot the movable jaw with respect to the shank. The actuation rod is detachably connected to the movable jaw part and is pivotable about its distal end through a pivot angle with respect to the shank. In order to prevent detaching the actuation rod from the movable jaw part, this pivot angle is limited to a maximum pivot angle by an articulated limitation element connected between the actuation rod and the shank.

### BACKGROUND OF THE INVENTION

1. Field of the Invention

The invention relates to a medical forceps including a shank having a distal end with a fixed jaw part, a movable jaw part which can pivot with respect to the fixed jaw part, and an actuation rod having a distal end which is axially movable with respect to the shank in order to pivot the movable jaw with respect to the shank; the actuation rod is detachably connected to the movable jaw part and is pivotable about its distal end through a pivot angle with respect to the shank.

2. Description of the Related Art

In the medical field, one applies forceps which are shaped for gripping or firmly holding tissue or objects, also under very difficult access conditions, such as in the field of arthroscopy. These forceps consist usually of a forceps jaw at the distal end of the forceps, of a handle at the proximal side, and of a shank located between the forceps jaw and the handle. The forceps jaw is of two parts, wherein mostly one jaw half is rigidly connected to the shank and a second jaw half is movably mounted. The opening and closure of the forceps jaw is effected via an axially adjustable actuation rod guided in the shank in that this rod displaces the movably mounted jaw half in a pivotally movable manner. With some forceps, the guiding of the pivotally movable jaw part is effected in an arc-shaped groove of the shank. The actuation rod is loosely suspended into a further arc-shaped groove of the pivotally movable jaw part. There is no rigid mechanical connections between the shank, the actuation rod and the movable jaw part. Forceps with this construction are hereinafter called jointless forceps.

This solution of a jointless forceps is known from the U.S. Pat. No. 4,712,545. Here the forceps shank in the longitudinal direction comprises a channel in which the actuation rod is guided in an open manner. This design leads to considerable hygienic problems since the forceps is difficult to clean, which may lead to an endangering of the patient, e.g. by way of the transition of germs. An adequate cleaning of the intermediate space between the shank and the actuation rod is only possible if the actuation rod is pivoted away to the outside out of the forceps shank and away from this. This pivoting movement from a certain pivot-out angle may lead to the movable jaw part and the actuation rod being detached from the forceps. The individual parts must then be assembled again by the user, which one may not expect of the medical personnel, or may even not at all be possible.

While the actuation rod with the forceps described above, on opening and closure of the forceps jaw, at its distal end pivots slightly up and down in the relief of the shank, a jointless forceps is introduced in the U.S. Pat. No. 5,366,477 with which this pivoting-out is avoided on account of a rotation point (fulcrum) at the distal end of the actuation rod. The actuation rod may be guided in a tubular shank in a straight line. This tube may be designed such that it is sealed off—or also rinsed, which reduces the above described hygienic problems. The disadvantage with this design is the further rotation point (fulcrum) since on account of small dimensions for a usual loading, a breakage of a component may easily occur. The actuation rod on account of the guiding in the closed shank tube is also dimensioned smaller than with an open forceps jaw so that here too stability problems may occur.

### SUMMARY OF THE INVENTION

Proceeding from this it is the object of the invention to create a medical forceps which has a stability corresponding to the demands of use and is easy and unproblematic to clean.

The medical forceps according to the invention is a jointless forceps with a shank, at least one movable jaw part arranged at the distal end of the shank and an actuation rod which by way of pivoting is detachably connected to the jaw part and for moving the jaw part is guided on the shank in an axially displaceable manner, and may be pivoted about its distal end outwards with respect to the shank, wherein the maximum pivot angle between the actuation rod and the shank is limited by way of a limitation element. The forceps jaw may be opened by way of axial displacement of the actuation rod. For this, the movable jaw part is connected in a pivotally movable manner to the shank and to the distal end of the actuation rod. For dismantling the forceps, the actuation rod is pivoted to such an extent about the pivot connection between the jaw part and the actuation rod that the actuation rod is disengaged from the pivot connection. For cleaning, the actuation rod may likewise be pivoted open about the pivot connection between the jaw part and the actuation rod, so that it may be removed from the shank and the space between the actuation rod and the shank may be cleaned. At the same time the limitation element limits the pivot angle of the actuation rod to an angle at which the actuation rod at the pivot connection remains engaged with the jaw part.

The movable jaw part is for example guided in a pivotally movable manner in at least one semicircular guide path of the shank via an arc-shaped shoulder of the jaw part, the shoulder engaging in this guide path. A lateral, arc-shaped shoulder of the actuation rod corresponding to the groove engages in a semicircular groove of the jaw part which is open to the top and which is arranged offset to the guide path of the shank by 180°. For opening the forceps, the actuation rod is displaced axially in the direction of the handle. By way of this the shoulder of the actuation rod, which engages in the groove, is moved partly out of the groove while the jaw part is simultaneously pivoted out guided via the guided path of the shank. If the actuation rod is pivoted open about its distal end for cleaning, the shoulder of the actuation rod is moved out of the groove on the jaw part until on reaching a certain pivot-open angle, the linkage between the movable jaw part and the actuation rod is lost. The actuation rod is then separated from the forceps. Simultaneously the movable jaw part slides out of the guide path of the shank and is thus detached from the forceps. This falling-apart of the forceps is prevented by way of the limitation element between the actuation rod and the shank. The limitation element limits the pivot-open angle of the actuation rod in a manner such that the linkage between the actuation rod and the jaw part is not lost even when the actuation rod has been pivoted open. This permits the forceps to be cleaned without any problem and without a danger of the forceps falling apart.

An articulated connection between the actuation rod and the shank is provided as a limitation element. The articulation permits the pivoting-open of the actuation rod, wherein the pivot-open angle is limited by the length of this articulated connection and the position of its connection to the actuation rod and the shank. Furthermore the articulation also permits the retraction (folding-in) of the limitation element so that in the condition of not being pivoted open it may assume a compact arrangement.

The limitation element is usefully coupled on the shank and on the actuation rod in an articulated manner. By way of this also rigid limitation elements in the form of a lever or of a distance piece may be used.

Preferably the limitation element at its first end is rotatably connected to the shank and to the actuation rod via a first pin. The pin forms the rotation point about which the limitation element is rotated on its pivoting-open movement relative to the shank or to the actuation rod.

At its second end the limitation element advantageously has a slot extending in the longitudinal direction of the limitation element, in which a second pin connected to the shank or to the actuation rod is guided. During the pivoting-open of the actuation rod the distance of the linkage points of the limitation element to the shank and the actuation rod increases. This change in distance is compensated by the guiding of the second pin in the slot. Furthermore the slot ensures the displacability of the actuation rod for opening and closing the forceps jaw. For this, the slot in the axial direction has a length which is larger than the displacement path of the actuation rod.

For example, the rotatable connection of the limitation element is located via the first pin on the actuation rod, while the second pin for guiding the limitation element is attached on the shank in its opening.

Advantageously the actuation rod or the shank comprises a recess in which the limitation element is arranged in the non-pivoted condition of the actuation rod. In this manner the arrangement of the limitation element is possible with the same outer dimensions of the forceps. Preferably the actuation rod in the non-pivoted condition is guided in a channel of the shank running in the longitudinal direction. By way of the recess for the limitation element it is not necessary to enlarge the cross section of this channel or the cross section of the shank.

Advantageously a relief is provided on the actuation rod in the region of the recess, and is located at the side facing the shank and in the non-pivoted condition of the actuation rod accommodates the second pin. This permits a compact arrangement of the actuation rod on the shank since it may be applied directly onto the shank. The relief is shaped elongate in the longitudinal axis of the actuation rod. At the same time the length and position of the relief is fixed by way of the axial movement of the actuation rod on opening and closure of the forceps jaw. Thus the length of the relief corresponds at least to the maximum possible axial displacement path of the actuation rod. The position of the relief is selected such that with each relative position of the actuation rod to the second pin, in particular in the condition of the largest possible displacement in the direction of its distal and its proximal end, the pin engages into the relief.

The limitation element preferably comprise an abutment which with a maximum pivot angle of the actuation rod prevents a further rotation of the actuation rod relative to the limitation element. By way of this the pivoting of the actuation rod out of the connection to the jaw part is limited so that this may not detach from the actuation rod and thus also not from the shank.

Usefully for this, the recess on the actuation rod comprises an abutment on which an abutment surface of the limitation element bears given the maximum permissible angular bending of the actuation rod. By way of this, a further rotation of the actuation rod in the pivoting-open direction about the linkage point on the limitation element is prevented.

On pivoting open the actuation rod, the limitation element connected to this also pivots out. So that its end which is applied onto the shank does not collide with this and thus blocks a further pivoting open of the actuation rod, the shank has an elongate opening in the bottom of the channel. That end of the limitation element which is faces the shank moves into this opening on pivoting.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

FIG. 1 is a partly sectioned lateral view of the distal end of a medical forceps with a pivoted open actuation rod; and

FIG. 2 is a partly sectioned lateral view of the distal end of the medical forceps according to FIG. 1, with an actuation rod which is not pivoted open.

### DETAILED DESCRIPTION OF THE PREFERRED EMBODIMENT

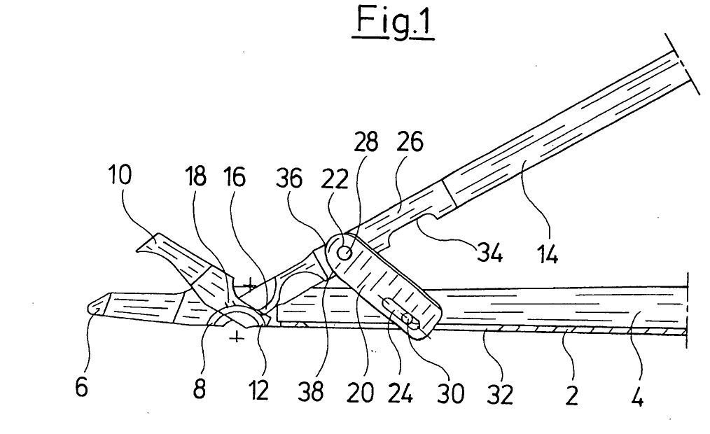

FIG. 1 shows the distal end of the medical forceps. A rigid jaw part 6 is rigidly connected at the distal end of a shank 2 which over its whole length has a channel 4 open to the top. In the region of the connection of the jaw part 6, the shank 2 has a semicircular guide path 8 which passes through its lower side twice and in which a pivotally movable jaw part 10 comprising a semicircular shoulder 12 corresponding to the guide path 8 is mounted via this shoulder 12, so that the jaw part 6 maybe pivoted on the semicircular guide path 8.

An actuation rod 14 at its distal end, via a semicircular shoulder 16 whose apex points in the direction of the shank 2, is arranged in a semicircular groove 18 of the jaw part 10, this groove corresponding to the shoulder 16.

A rigid limitation element 20 is located between the shank 2 and the actuation rod 14. This element in the vicinity of its first end has a circular hole 22 and in the vicinity of its second end has a slot 24, wherein the slot 24 is directed parallel to the longitudinal axis of the limitation element 20.

The actuation rod 14 on its longitudinal side comprises a recess 26 which extends over the whole width of the longitudinal side in the direction of its longitudinal axis. A pin 28 is arranged in one end of the recess 26. The limitation element 20 is rotatably linked to this pin 28 via its hole 22. The pin 28 forms the rotation point about which the limitation element 20 is rotated relative to the shaft 2 or to the actuation rod 14 with its pivoting movement.

A second pin 30 is arranged in the channel 4 of the shank 2 in the vicinity of its lower side. This is accommodated by the slot 24 of the limitation element 20 and on pivoting open the actuation rod 14, is guided in the slot 24 of the limitation element 20. Due to the guiding of the pin 30 in the slot 24 the distance change of the linkage points of the limitation element 20 to the shank 2 and the actuation rod 14 are compensated during the opening and closure pivoting of the actuation rod 14.

At the distal end, the shank 2 on its lower side comprises an elongate opening 32 arranged parallel to the longitudinal axis of the shank. This in the pivoted open condition of the actuation rod 14 is penetrated by the shaft-side end of the limitation element 20. The opening 32 is so long that in the condition of the actuation rod 14 not being pivoted open, this opening may accommodate a lower region of the limitation element 20, the region facing the shank 2, over the whole length of the element and furthermore may permit displacement of the limitation element 20 due to the axial movement of the actuation rod 14 on opening and closure of the forceps jaw.

In the region of the recess 26 of the actuation rod 14, on that side of the actuation rod 14 which faces the shank 2 there is provided a relief 34 which assumes the whole width of the actuation rod 14 in a direction transverse to the recess 26. The relief 34 in the condition of the actuation rod 14 not being pivoted open accommodates the pin 30 and is so long that it permits the axial movements of the actuation rod 14 necessary for opening and closing the forceps jaw.

Furthermore the recess 26 of the actuation rod 14 at its distal end has an abutment surface 36. Likewise the limitation element 20 at its end coupled onto the actuation rod 14 has an abutment 38. On pivoting open the actuation rod 14 the abutment surface 36 and the abutment 38 face one another and with a maximal pivot-open angle of the actuation rod 14 lie on one another in a planar manner. This prevents a further rotation of the actuation rod 14 in the pivot-open direction relative to the actuation element 20 and thus a pivoting of the actuation rod 14 out of the guide of the pivotally movable jaw part 19, i.e. of the shoulder 16 out of the groove 18.

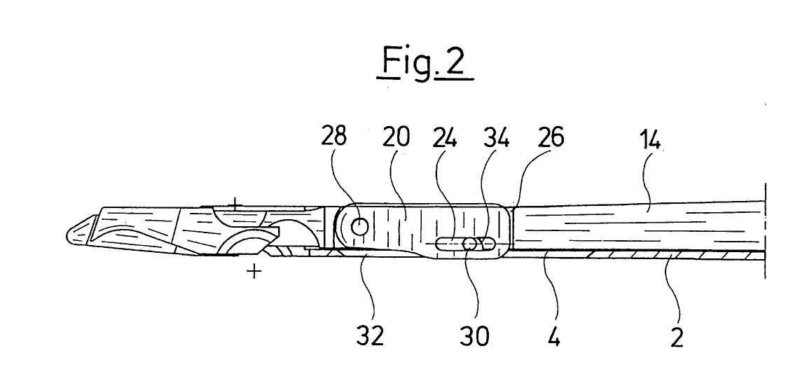

In FIG. 2 the distal end of the medical forceps according to FIG. 1 is represented with the actuation rod 14 not pivoted out. The actuation rod 14 is located completely in the channel 4 of the shank 2. The limitation element 20 is rotated about the fulcrum of the pin 28 so that its longitudinal axis runs parallel to the longitudinal axis of the actuation rod 14. It is also arranged in the channel 4, wherein it is completely accommodated in the relief 26 of the actuation rod 14. The lower side of the limitation element 20 penetrates the opening 32 on the lower side of the shank 2, which is provided for this. The second pin 30 which is connected to the shank 2 lies in the relief 34 of the actuation rod 14.

FIG. 2 shows a forceps with a closed forceps jaw. In this condition the actuation rod 14 is displaced maximally in the direction of the forceps jaw, i.e. distally. For pivoting open the jaw part 10 or for opening the forceps jaw, the actuation rod 14 is moved in the direction of its proximal end. The opening 32 of the shank 2, as well as the slot 24 of the limitation element 20 and the relief 34 of the actuation rod 14 each have a length which is larger than the maximum displacement path of the actuation rod 14 and thus permits the displacement.

The invention is not limited by the embodiments described above which are presented as examples only but can be modified in various ways within the scope of protection defined by the appended patent claims.

1. A medical forceps comprising:
a shank having a distal end with a fixed jaw part;
a movable jaw part which can pivot with respect to said fixed jaw part;
an actuation rod having a distal end which is axially movable with respect to the shank in order to pivot the movable jaw with respect to the shank, said actuation rod being detachably connected to said movable jaw part and being pivotable about its distal end through a pivot angle with respect to said shank; and
a limitation element which limits the pivot angle to a maximum pivot angle in order to prevent detaching the actuation rod from the movable jaw part.

2. The medical forceps of claim 1 wherein said limitation element comprises an articulated connection between the actuation rod and the shank.

3. The medical forceps of claim 1 wherein the limitation element is articulately coupled to the shank and to the actuation rod.

4. The medical forceps of claim 3 wherein said limitation element has a first end which is rotatably connected to one of said shank and said actuation element by a first pin.

5. The medical forceps of claim 4 wherein said limitation element has a second end provided with a slot which receives a second pin fixed to the other of said shank and said actuation element.

6. The medical forceps of claim 5 wherein said actuation rod comprises a recess which receives the limitation element when the pivot angle is zero.

7. The medical forceps of claim 6 wherein said actuation rod is provided with a relief which receives the second pin when the pivot angle is zero.

8. The medical forceps as in claim 1 wherein the limitation element comprises an abutment surface which limits rotation of the actuation rod with respect to the limitation element, thereby establishing the maximum pivot angle.

9. The medical forceps of claim 6 wherein the recess comprises an abutment which the limitation element abuts to limit rotation of the actuation rod with respect to the limitation element, thereby establishing the maximum pivot angle.

10. The medical forceps of claim 1 wherein the shank comprises an elongate opening which receives part of said limitation element during pivoting of said actuation rod with respect to said shank.
